(19) United States
(12) Patent Application Publication
(10) Pub. No.: US 2005/0003012 A1
(43) Pub. Date: Jan. 6, 2005

(54) Gel matrix consisting of polyacrylic acid and polyvinyl pyrrolidone

(76) Inventors: Karl-Heinz Woller, Hamburg (DE); Jens Nierle, Hamburg (DE)

Correspondence Address: GREENBLUM & BERNSTEIN, P.L.C., 1950 ROLAND CLARKE PLACE, RESTON, VA, 20191, US

(21) Appl. No.: 10/486,356

(22) PCT Filed: Aug. 21, 2002
(86) PCT No.: PCT/EP02/09311

(30) Foreign Application Priority Data
Sep. 1, 2001 (DE) 10142918.5

(51) Int. Cl. A61K 9/14; C08L 39/04

(52) U.S. Cl. 424/487; 525/205

(57) ABSTRACT

A self-adhesive polyacrylic acid-based gel matrix. The gel matrix comprises a homopolymer and/or copolymer of vinyl pyrrolidone as crosslinker for the polyacrylic acid.

The invention relates to a self-adhesive gel matrix, in particular a monolithic gel matrix, which is based on polyacrylic acid and comprises polyvinylpyrrolidone (PVP) as crosslinking agent. The gel matrix can be doped with hydrophilic, and hydrophobic with a suitable solubilizer, active ingredients for the cosmetic and/or pharmaceutical treatment of the skin or systemic administration of medicaments.

The production of gel matrices from polyacrylates has been known for many years and is described for example in EP 0 507 160, JP 11-228340 and JP 04178323. Gel matrices are employed inter alia as adhesive base and active ingredient reservoir in transdermal systems. An embodiment of transdermal systems which is well described in the specialist literature is represented by matrix systems or monolithic systems in which the medicinal substance is incorporated directly into the pressure-sensitive adhesive. Such a pressure-sensitive adhesive, active ingredient-containing matrix is provided, in the product ready for use, on one side with a backing which is impermeable to the active ingredient, and on the opposite side there is a backing film which is provided with a separating layer and is removed before application to the skin (kleben&dichten, No. 42, 1998, pp. 26 to 30). However, the described matrices usually have only low intrinsic adhesiveness, so that an additional adhesive application aid is necessary for permanent fixation to the skin. Or the systems have sufficient adhesiveness, specifically to moist skin (buccal patch), but cannot be detached again completely when required because of inadequate cohesiveness.

Polyacrylic acid must be crosslinked to form a gel of defined structure. The nature of the crosslinker makes a crucial contribution to the structure of the resulting gel in this case. The usual crosslinking agents may in this connection be metal ions (e.g. Al³⁺ ions), or organic compounds. Crosslinking with aluminum salts proceeds via coordination of the oxygen functions of the polyacrylic acid to the Al³⁺ ions. A very close-mesh gel with high viscosity is formed, it being possible to control the viscosity of the gel only via the amount of crosslinker (handbook of pressure sensitive adhesive technology, page 458 et seq., 1999).

JP 11-228340 discloses polyacrylic acid-based gels which utilize Al³⁺ compounds as crosslinkers. Use of the obligatorily necessary aluminum compound as crosslinking agent is limited because, otherwise, the physical properties of the gel deteriorate. The gel becomes too hard if the content of aluminum crosslinker is too high.

Further examples of crosslinking with multivalent metal ions are known from the literature, e.g. U.S. Pat. No. 3,900,610 (zinc salts), U.S. Pat. No. 3,770,780 or U.S. Pat. No. 3,790,533 (titanium compounds). Ionic crosslinking with metal ions leads to hard, viscous and low-tack polymer gels (handbook of pressure sensitive adhesive technology, page 458 et seq., 1999).

A further problem in the crosslinking of polyacrylic acid to give a self-adhesive gel is that a gel once produced with defined physical properties, viscosity, tack etc., must display the same defined properties in a later production process. This reproducibility is costly to achieve, if at all, with the currently known crosslinking technologies.

EP 303445 discloses a patch with monolithic gel matrix based on water-soluble polymers. The obligatorily necessary components which are provided are clebopride or a pharmaceutically acceptable salt thereof as active ingredient, water, water-absorbing agents and water-soluble polymers. The skilled worker is able to select water-soluble polymers from a number of known polymers such as polyvinyl alcohol, gelatin, polyacrylic acid, sodium polyacrylates, methylcellulose, carboxymethylcellulose, polyvinylpyrrolidone, gum and other crosslinkable polymers, and mixtures thereof.

A difference from the gel matrix of the invention is that PVP is disclosed as one possibility for the water-soluble polymer, but not as crosslinker for polyacrylic acid-based self-adhesive gels. A problem also described in EP 303445 is the reduction in viscosity and loss of adhesiveness of the polymers through changing the composition, especially the crosslinking agent.

It is an object of the invention to avoid the disadvantages known in the prior art and provide a self-adhesive gel matrix whose tack, cohesiveness and viscosity can be adjusted individually for the particular area of application. The particular object of the invention is to provide a self-adhesive gel matrix for transdermal systems which combines the adhesiveness necessary for a monolithic patch application with the appropriate cohesiveness.

This object is achieved by a gel matrix as set forth in claim 1. The dependent claims relate to advantageous further developments of the gel matrix.

In the gel matrix of the invention, the crosslinking of the polyacrylic acid is carried out with the aid of polyvinylpyrrolidone (PVP).

The crosslinking proceeds via formation of a quaternary ammonium salt of PVP. This type of crosslinking leads to organic salts which, in contrast to known metal salts as crosslinking agents, are linked via the hydroxy functions of the polyacrylic acid molecules. As with the metal salts, the reaction is reversible and can be reversed through addition of water or acids. Surprisingly, the viscosity of the resulting gel can be controlled not only via the amount of crosslinker but also via the molecular weight of the PVP. In this connection, high molecular weights lead to gels of low viscosity and low molecular weights lead to gels of high viscosity and adhesiveness. The advantage of the mode of crosslinking according to the invention is thus the targeted production, via the parameters of PVP content and PVP molecular weight, of gel matrices whose tack, cohesiveness and viscosity can be adjusted individually for the particular area of application.

This effect of the influence of the PVP molecular weight on the viscosity and adhesiveness of the gel matrix is attributable to the following realization: the number of pyrrolidone subunits per macromolecule is distinctly higher in long-chain PVP than in short-chain PVP. This results in an increase in reactions of identical reactants with one another, because the macromolecules can easily align to form bundles. These reactions do not lead to a formation of linkage points with a plurality of polyacrylic acid molecules. Therefore only a few crosslinks are formed with other polyacrylic acid molecules, and thus only a few, large meshes are formed. This circumstance leads to a loosely linked gel of low viscosity. In contrast thereto, in the case of short-chain PVP, because of the greater mobility and the smaller tendency of the molecules to align to form strands, more linkages with different polyacrylic acid molecules are formed and lead to a narrower mesh width and lower flexibility and viscosity of the gel.

The viscosity of the gels can additionally be controlled via other factors. Thus, for example, the amount of PVP contributes to determining the structure of the gel. If a saturation point is exceeded, competing reactions of the free PVP with those already crosslinked occur. These competing reactions lead to crosslinkage points being broken open in favor of unlinked aggregates of polyacrylic acid and the excess PVP molecules. The consequence of this supersaturation is a decrease in the total number of linkage points and thus a decrease in the gel viscosity. A further possibility which can be utilized for controlling the gel viscosity is to add protic solvents (e.g. water, alcohols, amines, thiols) or organic proton donors (carboxylic acids e.g. salicylic acid) or inorganic agents (e.g. Lewis acids). Specifically suitable for this are compounds from the families of tertiary polyamines and polyamides. In each of these cases, addition of the agents contributes to reducing the coordination points either on the polyacrylic acid or on the PVP. This reduces the number of potential linkage points for forming gel meshes, thus directly influencing the viscosity of the gel.

The resulting gel properties of the matrices can additionally be influenced via the molecular weight, degree of substitution and degree of crosslinking of the polyacrylic acid employed.

To produce particular application properties, the gel matrices are mixed with the appropriate plasticizers, solubilizers, penetration enhancers, fillers and/or other known additives.

Polyacrylic acid is employed as gel base. Polyacrylates are gel-forming polymers which can be used advantageously for the purposes of the present invention. Polyacrylates which are advantageous according to the invention are acrylates/alkyl acrylate copolymers, especially those chosen from the group of so-called carbomers or Carbopols (Carbopol® is actually a registered trademark of the B.F. Goodrich company). The acrylate/alkyl acrylate copolymer(s) advantageous according to the invention have the following structure in particular:

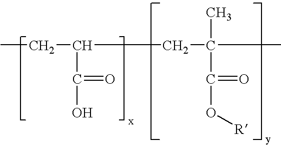

In this, R′ is a long-chain alkyl radical and x and y are numbers which symbolize the respective stoichiometric content of the respective comonomers.

Particularly preferred according to the invention are acrylate copolymers and acrylate/alkyl acrylate copolymers, which are obtainable under the proprietary names Carbopol® 1382, Carbopol® 981 and Carbopol® 5984 from the B.F. Goodrich company, preferably polyacrylates from the group of the Carbopols of the 980, 981, 1382, 2984, 5984 types and particularly preferably carbomer 2001.

Also advantageous are copolymers of C₁₀₋₃₀-alkyl acrylates and one or more monomers of acrylic acid, of methacrylic acid or esters thereof, which are crosslinked with an allyl ether of sucrose or an allyl ether of pentaerythritol.

Polyacrylic acid and/or copolymers thereof are preferably employed in an amount of 5-55% by weight, particularly preferably between 5-30% by weight. All percentage data are based in this connection on contents of gel matrix by weight, unless the opposite is indicated.

The crosslinker employed is polyvinylpyrrolidone (PVP), e.g. Luviskol from BASF, preferably in an amount of 0.25-60% by weight, particularly preferably between 1-30% by weight. It is also possible to the same extent to employ PVP copolymers such as, for example, vinylpyrrolidone/vinyl acetate (povidone acetate; Kollidon VA 64), terpolymers based on vinylpyrrolidone and acrylic acid or methacrylic acid and esters thereof (Luviflex VBM 35), copolymers of vinylpyrrolidone and vinylimidazolium methochloride (Luviquat brands) as so-called PVP crosslinking agent.

Further constituents of the gel which can be employed are polyalcohol or polyalcohols, e.g. 1,2-propanediol, glycerol, and/or water, preferably in an amount of 5-90% by weight, particularly preferably between 5-45% by weight.

Further constituents of the gel matrix may be stabilizers, e.g. polyethylene glycols (Lutrol E400, E600 from BASF) in an amount of 0-50% by weight, preferably 0-30% by weight, neutralizers, e.g. tromethamol, triethanolamine and/or dexpanthenol, in an amount 0-30% by weight, preferably 0-15% by weight, filler(s), e.g. silica, micronized cellulose and/or gelatin, in an amount of 0-30% by weight, preferably 3-15% by weight, and natural active ingredient(s), e.g. menthol, jojoba oil, ibuprofen, benzyl nicotinate and/or capsaicin, in an amount of 0-35% by weight, preferably 0-15% by weight.

These gel matrices are produced without solvent, preferably at room temperature, in commercially available kneaders or suitable extruders. The polyacrylic acid-based gel matrix of the invention combines the necessary adhesiveness with the appropriate cohesiveness for a monolithic patch application. For application as patches, the gel matrices are pressed, rolled or the like as layer onto a separating medium of paper, film or the like, and laminated on the reverse side with any desired backing material.

The gel matrix of the invention is particularly advantageously applied to a flexible cover layer, especially for use as patch. An appropriate patch is composed of a backing such as films, nonwovens, wovens, foams etc., the adhesive matrix and covering film, covering paper or separating paper to protect the adhesive matrix before use of the patch.

In a further preferred embodiment of the invention, polymer films, nonwovens, wovens and combinations thereof are employed as backing. Available for selection as backing materials are, inter alia, polymers such as polyethylene, polypropylene and polyurethane or else natural fibers.

In summary, it can be stated that all rigid and elastic sheet-like structures of synthetic and natural raw materials are suitable as backing materials. Preferred backing materials are those which can be employed in such a way that they comply with the properties of the properly functioning dressing. Examples listed are textiles such as wovens, knits, lace, nonwovens, laminates, nets, films, foams and papers. In addition, these materials can be pretreated or aftertreated. Conventional pretreatments are corona and hydrophobias; customary aftertreatments are calendering, heat treatment, laminating, punching and lining.

It is particularly advantageous for the carrier material to be sterilizable, preferably γ (gamma) sterlizable.

Said properties of the adhesive matrix suggest in particular the use for medical products, especially patches, medical fixings, wound coverings, orthopedic and phlebological bandages and dressings.

Finally, the gel matrix can be lined with an adhesive-repellent backing material such as siliconized paper or be provided with a wound pad or a cushion. The patch of the invention is normally covered on its side which has a self-adhesive finish and later faces the skin over its whole width until used with an adhesive-repellent backing material. This protects the self-adhesive layer from the gel matrix adhesive composition, which is well tolerated by skin and has preferably been applied by a transfer process, and additionally stabilizes the whole product. The covering can be designed in a known manner to be in one piece or, preferably, in two parts.

Further embodiments may be configured so that a second matrix with higher active ingredient solubility is present as reservoir between the reverse side of the matrix and the covering backing. This might be, instead of a second matrix and backing, also a thermoformed film with pure active ingredient.

On part (e.g. at the edge) of the adhesive side of the matrix there is a second matrix with high adhesiveness for additional fixation, but inadequate active ingredient solubility.

The active ingredient-free matrix is located between two non-anchoring films and is used for the fixation of electrodes etc., or, because of the water uptake capacity, with an appropriate geometry of colostomy/ileostomy bags. The active ingredient-free matrix may also serve (with or without wound pad) as adhesive layer for a simple wound/adhesive plaster.

The following examples illustrate the invention without restricting it. The gel matrices of the invention are listed in the following table.

### EXAMPLES 1 to 9

| Constituents/Example | 1 | 2 | 3 | 4 | 5 | 6 | 7 | 8 | 9 |
|---|---|---|---|---|---|---|---|---|---|
| Polyacrylic acid | 22.5% | 18.0% | 22.5% | 22.5% | 22.5% | 22.5% | 22.5% | 21.0% | 21.0% |
| Polyvinylpyrrolidone | 3.5% | 3.5% | 3.5% | 3.5% | 3.5% | 3.5% | 3.5% | 3.5% | 3.5% |
| PVP 25 | | | | | | | | | |
| Propanediol | 36.0% | 43.5% | 36.5% | 37.5% | 35.5% | 25.5% | 40.0% | 35.5% | 35.5% |
| Polyethylene glycol | 17.0% | 23.5% | 17.0% | 6.5% | 20.0% | 12.0% | 21.0% | 20.0% | 20.0% |
| Silica | 8.0% | 5.0% | 10.0% | 10.0% | 11.5% | 11.5% | 10.0% | 10.0% | 10.0% |
| Dexpanthenol | 5.0% | 5.0% | 5.0% | 5.0% | 5.0% | 5.0% | — | 5.0% | 5.0% |
| Jojoba oil | 5.0% | 0.5% | 5.0% | 5.0% | — | — | — | — | — |
| Salicylic acid | 3.0% | 1.0% | — | — | — | — | 3.0% | — | — |
| Benzyl nicotinate | — | — | 0.5% | — | — | — | — | — | — |
| Glycol salicylate | — | — | — | 10.0% | — | 10.0% | — | — | — |
| Menthol | — | — | — | — | 2.0% | — | — | — | — |
| Peppermint oil | — | — | — | — | — | 10.0% | — | — | — |
| Ibuprofen | — | — | — | — | — | — | — | 5.0% | — |
| Tea tree oil | — | — | — | — | — | — | — | — | 5.0% |

### EXAMPLES 10 to 18

| Constituents/Examples | 10 | 11 | 12 | 13 | 14 | 15 | 16 | 17 | 18 |
|---|---|---|---|---|---|---|---|---|---|
| Polyacrylic acid | 5.0% | 10.5% | 10.5% | 21.0% | 21.0% | 21.0% | 10.5% | 11.7% | 18.0% |
| Polyvinylpyrrolidone | 30.0% | 3.5% | 3.5% | 3.5% | 3.5% | 3.5% | 3.5% | 3.9% | 3.5% |
| Propanediol | — | — | 5.0% | 35.5% | 35.5% | 35.5% | — | — | 58.5% |
| Polyethylene glycol | — | — | — | 15.0% | 20.0% | 20.0% | 20.0% | 22.1% | — |
| Diethylene glycol | 25.0% | — | — | — | — | — | — | — | — |
| Glycerol | — | 72.47% | 77.0% | — | — | — | 46.0% | 51.1% | 10.0% |
| Water | 15.0% | — | — | — | — | — | — | — | — |
| Polyoxyethylene 20 | — | 10.0% | — | — | — | — | — | — | — |
| sorbitan monolaurate | | | | | | | | | |
| Isopropyl myristate | — | — | — | 5.0% | — | — | — | — | — |
| Silica | 15.0% | — | — | 10.0% | 10.0% | 10.0% | 10.0% | — | — |
| Gelatin | — | 3.5% | 3.5% | — | — | — | — | — | — |
| Dexpanthenol | — | — | — | 5.0% | 5.0% | 5.0% | 5.0% | 5.6% | 5.0% |
| Urea | 10.0% | — | — | — | — | — | — | — | — |
| Vitamin A palmitate | — | 0.03% | — | — | — | — | — | — | — |
| Capsicum extract | — | — | 0.5% | 5.0% | — | — | — | — | — |
| Clotrimazole | — | — | — | — | 5.0% | — | — | — | — |
| Lidocaine HCl | — | — | — | — | — | 5.0% | — | — | — |
| Ibuprofen | — | — | — | — | — | — | 5.0% | 5.6% | 5.0% |

For application as patch, all the gel matrices listed by way of example have been rolled as layer onto a separating medium (backing) of paper and film, and their adhesive property and cohesiveness have been assessed organoleptically. All the patches of the invention differ from patches with known gels in providing sufficiently good adhesiveness and appropriate cohesiveness, so that all the patches can be detached from the skin without residues.

### EXAMPLES 19 to 25

In accordance with example 1, the following polyvinylpyrolidones or mixtures thereof are employed in place of polyvinylpyrrolidone PVP 25.

| | | Average molecular |
|---|---|---|
| Example | | weight [g/mol] |
| 19 | PVP 12 | ca. 2 500 |
| 20 | PVP 17 | ca. 11 500 |
| 21 | PVP 25 | ca. 25 000 |
| 22 | PVP 30 | ca. 40 000 |
| 23 | PVP 90 | ca. 700 000 |
| 24 | PVP 12 and PVP 30, 1:1 | ca. 21 250 |
| 25 | PVP 25 and PVP 90, 1:1 | ca. 370 000 |

Example 19, 20, 21 and 24 led to gels whose adhesiveness could be assessed as good or very good and whose cohesiveness could be assessed as adequate. Example 22, 23 and 25 led to gels of low viscosity and less adhesiveness. The advantage of the mode of crosslinking according to the invention is thus the specific production, via the parameters of VP content and PVP molecular weight, of gel matrices whose tack, cohesiveness and viscosity can be adjusted individually to the particular area of application.

The advantageous properties of the gel matrices of the invention in transdermal systems are thus impressively confirmed.

1-19. (canceled)

20. A self-adhesive polyacrylic acid-based gel matrix, wherein the gel matrix comprises at least one of a homopolymer and a copolymer of vinyl pyrrolidone as a crosslinker of the polyacrylic acid.

21. The gel matrix of claim 20, wherein the polyacrylic acid comprises an acrylates/alkyl acrylate copolymer.

22. The gel matrix of claim 20, wherein the at least one of a homopolymer and a copolymer of vinyl pyrrolidone is present as a mixture of polymers of different average molecular weights.

23. The gel matrix of claim 20, wherein the at least one of a homopolymer and a copolymer of vinyl pyrrolidone comprises at least one polymer having an average molecular weight in a range of from 2,500 to 700,000 g/mol.

24. The gel matrix of claim 20, wherein the gel matrix comprises from 5% to 55% by weight of at least one of a homopolymer and a copolymer of acrylic acid.

25. The gel matrix of claim 24, wherein the gel matrix comprises up to 30% by weight of the at least one of a homopolymer and a copolymer of acrylic acid.

26. The gel matrix of claim 20, wherein the gel matrix comprises from 0.25% to 60% by weight of at least one of a homopolymer and a copolymer of vinyl pyrrolidone.

27. The gel matrix of claim 26, wherein the gel matrix comprises from 1% to 30% by weight of the at least one of a homopolymer and a copolymer of vinyl pyrrolidone.

28. The gel matrix of claim 20, wherein the gel matrix comprises from 5% to 30% by weight of at least one of a homopolymer and a copolymer of acrylic acid, and from 1% to 30% by weight of at least one of a homopolymer and a copolymer of vinyl pyrrolidone.

29. The gel matrix of claim 20, wherein the gel matrix further comprises at least one polyalcohol.

30. The gel matrix of claim 29, wherein the gel matrix comprises at least one of propanediol, polyethylene glycol and glycerol.

31. The gel matrix of claim 29, wherein the at least one polyalcohol is present in a concentration of from 5% to 90% by weight.

32. The gel matrix of claim 31, wherein the concentration is up to 45% by weight.

33. The gel matrix of claim 20, wherein the gel matrix further comprises at least one of a protic solvent, an organic proton donor and a Lewis acid.

34. The gel matrix of claim 33, wherein the gel matrix comprises at least one of water, an alcohol, an amine and a thiol.

35. The gel matrix of claim 33, wherein the gel matrix comprises salicylic acid.

36. The gel matrix of claim 20, wherein the gel matrix is doped with a hydrophilic active ingredient.

37. The gel matrix of claim 20, wherein the gel matrix is doped with a combination of a hydrophobic active ingredient and a solubilizer.

38. The gel matrix of claim 20, wherein the gel matrix is doped with at least one of dexpanthenol, jojoba oil, salicylic acid, benzyl nicotinate, glycol salicylate, menthol, peppermint oil, ibuprofen, tea tree oil, urea, vitamin A palmitate, capsicum extract, clotrimazole and lidocaine HCI.

39. The gel matrix of claim 20, which further comprises at least one of a plasticizer, a solubilizer, a penetration enhancer and a filler.

40. The gel matrix of claim 20, which comprises from 5% to 30% by weight of at least one of a homopolymer and a copolymer of acrylic acid, from 1% to 30% by weight of at least one of a homopolymer and a copolymer of vinyl pyrrolidone and from 5% to 45% by weight of at least one polyalcohol.

41. The gel matrix of claim 40, wherein the polyalcohol comprises at least one of propanediol, polyethylene glycol and glycerol.

42. The gel matrix of claim 41, wherein the gel matrix further comprises at least one of water, an alcohol, an amine and a thiol.

43. The gel matrix of claim 42, wherein the gel matrix is doped with at least one of a hydrophilic active ingredient and a combination of a hydrophobic active ingredient and a solubilizer.

44. The gel matrix of claim 41, wherein the gel matrix is doped with at least one of dexpanthenol,jojoba oil, salicylic acid, benzyl nicotinate, glycol salicylate, menthol, peppermint oil, ibuprofen, tea tree oil, urea, vitamin A palmitate, capsicum extract, clotrimazole and lidocaine HCI.

45. A transdermal system which comprises the gel matrix of claim 20.

46. The transdermal system of claim 45, wherein the transdermal system comprises a patch.

47. The transdermal system of claim 46, wherein the patch comprises a backing for the gel matrix and at least one of a covering film, a covering paper and a release paper.

48. A medical fixing which comprises the gel matrix of claim 20.

49. A wound covering which comprises the gel matrix of claim 20.

50. An orthopedic or phlebologic bandage which comprises the gel matrix of claim 20.

51. A dressing which comprises the gel matrix of claim 20.

52. A method for the cosmetic or medical treatment of skin, wherein the method comprises applying to at least parts of the skin the gel matrix of claim 20.

53. A method of controlling at least one of the viscosity and adhesiveness of a self-adhesive polyacrylic acid-based gel matrix, wherein the method comprises combining the gel matrix with at least one of a homopolymer and a copolymer of vinyl pyrrolidone as a crosslinker for the polyacrylic acid.

54. The method of claim 53, wherein the at least one of the viscosity and adhesiveness are controlled by an amount of the vinyl pyrrolidone polymer.

55. The method of claim 53, wherein the at least one of the viscosity and adhesiveness are controlled by an average molecular weight of the vinyl pyrrolidone polymer.

56. A process for producing a self-adhesive polyacrylic acid-based gel matrix, which process comprises mixing at least one of a homopolymer and a copolymer of acrylic acid with at least one of a homopolymer and a copolymer of vinyl pyrrolidone to crosslink the acrylic acid polymer.

57. The process of claim 56, wherein from 5% to 55% by weight of the at least one of a homopolymer and a copolymer of acrylic acid are employed.

58. The process of claim 57, wherein up to 30% by weight of the at least one of a homopolymer and a copolymer of acrylic acid are employed.

59. The process of claim 56, wherein from 0.25% to 60% by weight of the at least one of a homopolymer and a copolymer of vinyl pyrrolidone are employed.

60. The process of claim 58, wherein from 1% to 30% by weight of the at least one of a homopolymer and a copolymer of vinyl pyrrolidone are employed.

61. The process of claim 56, wherein further at least one of a polyalcohol and water is employed.

62. The process of claim 61, wherein the polyalcohol comprises at least one of propanediol, polyethylene glycol and glycerol.

63. The process of claim 61, wherein the at least one of a polyalcohol and water is employed in an amount of from 5% to 90% by weight.

64. The process of claim 63, wherein the amount is up to 45% by weight.

65. The process of claim 56, wherein further at least one of a hydrophilic active ingredient and a combination of a hydrophobic active ingredient and a solubilizer is employed.
